(19) United States
(12) Patent Application Publication
(10) Pub. No.: US 2015/0184936 A1
(43) Pub. Date: Jul. 2, 2015

(54) Absorbent Device

(71) Applicant: DYECOR LIMITED, Hampshire (GB)

(72) Inventor: Euan Eric Neil Thomas George Davidson, Hampshire (GB)

(21) Appl. No.: 14/415,223

(22) PCT Filed: Jul. 16, 2013
(86) PCT No.: PCT/GB2013/051904

§ 371 Date: Jan. 16, 2015

(30) Foreign Application Priority Data
Jul. 16, 2012 (GB) 1212660.3

(51) Int. Cl. F26B 5/16; A61F 13/42; B65D 30/06

(52) U.S. Cl. CPC F26B 5/16; B65D 29/04; A61F 13/42; A61F 2013/423

(57) ABSTRACT

An absorbent product has an elongate matted strip of absorbent material, including super absorber, within an enveloping container. This has a double skin bottom and a single, folded top skin and is heat sealed around its periphery to contain the absorbent material. The top skin has an elongate, double Z fold along its length. As the container absorbs moisture it expands from a flat form to a round shape. The super absorber causes tension in the skins of the container as it reaches its absorption limit which tears the self-adhesive strips. The folds unfold and the round shape slumps to expose inner webs. An indicator in the form off the words “FULL-CHANGE” printed in a bright colour repeatedly along the inner webs is provided to alert maintenance staff of the need to change filled absorbent products for fresh one.

The present invention relates to an absorbent product.

It is well known to contain absorbent material in a permeable enclosure to absorb moisture and allow it to be removed manually from the place where the water was collected by absorption. The absorbent material may be a so-called “super absorber”, which is able to absorb and retain larger amounts of water than ordinary cloths for instance.

A particular situation where absorbent products are deployed is in retail premises on the floor around chillers, refrigerators and deep freezes, all of which are prone to condensation forming outside and running onto the floor, which can be dangerous. Whilst such condensation absorbent products are effective whilst they have capacity to absorb, they do require replacement when they are full. This can be overlooked.

Other situations where absorbent products are used are for absorption of exudate as in wound dressings.

The object of the present invention is to provide an improved absorbent product.

According to the invention there is provided an absorbent product comprising:
absorbent material and
a permeable container of sheet material containing the absorbent material and having:
   a fold in the sheet material with an outer web of the material over-lying an inner web at the fold and
   an indicator on the inner web
      the arrangement being such that the outer web normally covers the indicator when the absorbent material has absorbed a determined amount of moisture, whereupon it swells, opening the fold and exposing the indicator.

Normally the fold will be a Z fold with an intermediate web also overlying the inner web. Two Z folds can be provided one beside the other, providing an inner web with a greater extent, as two adjacent inner webs, than the extent of the individual outer webs.

The indicator will normally be a printing on the inner web. The printing can extend onto the intermediate web, where the fold or folds are Z folds. Alternatively, the indicator can be a separate element, such as a ribbon lying along the inner web.

In certain embodiments, there is included:
frangible means holding the outer web over-lying the indicator on the inner web,
   the frangible means normally holding the outer web in its position covering the indicator until expansion of the absorbent material on absorption of causes the frangible means to break and expose the indicator.

The frangible means can be one or more adhesive elements applied to the outer web or webs to hold the fold or folds closed. Alternatively, the frangible means can be adhesive material on the inner web and/or indicator ribbon. Again the frangible means can be a series of tacks of arranged to hold the fold closed.

The permeable container can be formed completely of non-woven fabric or of perforate plastics material film or a combination of this fabric and film. In the preferred embodiment, a top is of non-woven fabric having the fold and a bottom is of perforate film, the better to retain absorbent material. The perforate film can be provided alone or as an outer layer to an inner layer of non-woven fabric.

The bottom can have applied to it adhesive or non-slip material for retaining the absorbent product in a use position.

In another embodiment, a self-adhesive web can extend out from the edges of the container for its securement in a use position. This is particularly in use for absorbing exudate from a region covered by the container, such as a wound or a leak. In the latter case, the container could be installed where a leak may occur. Occurrence is indicated by exposure of the indicator on absorption.

The absorbent material is or can include super-absorber. A biocidal agent may be provided with the absorbent material. The absorbent material and/or biocidal agent can be provided loose, but is preferably provided as an integrated strip of absorbent material enclosed within the container.

To help understanding of the invention, a specific embodiment thereof will now be described by way of example and with reference to the accompanying drawings, in which:

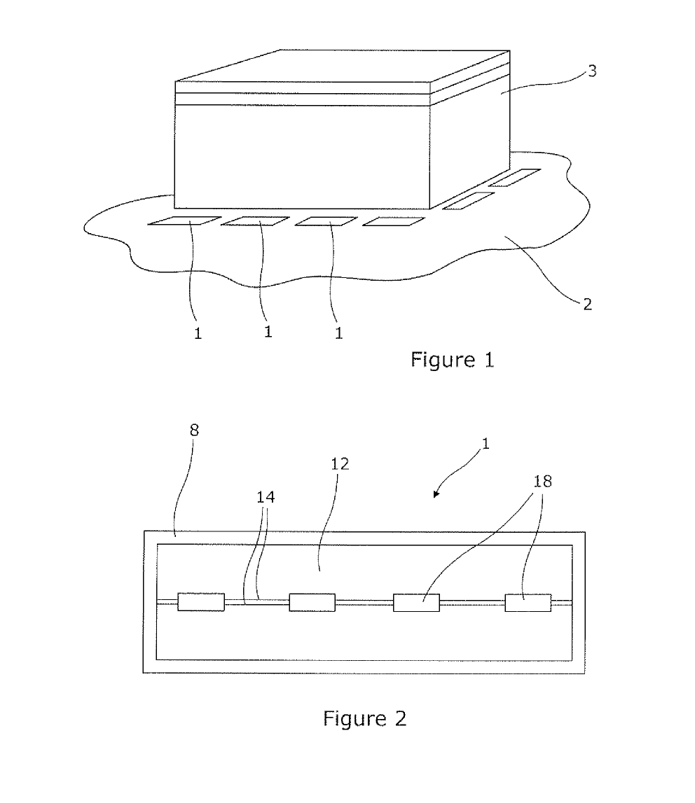

FIG. 1 is a perspective view of a freezer prone to condensation with absorbent products of the invention set out around it;

FIG. 2 is a plan view of a fresh one of the absorbent products of the invention;

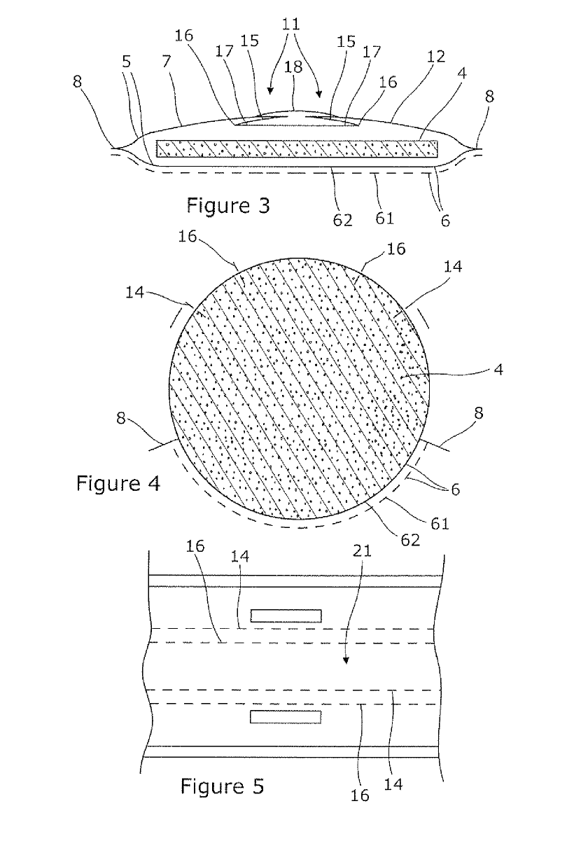

FIG. 3 is a cross-sectional end view of the product of FIG. 2;

FIG. 4 is a similar cross-sectional view of the product after absorbing its fill of moisture;

FIG. 5 is a plan view of the full product with its “FULL-CHANGE” indicator exposed;

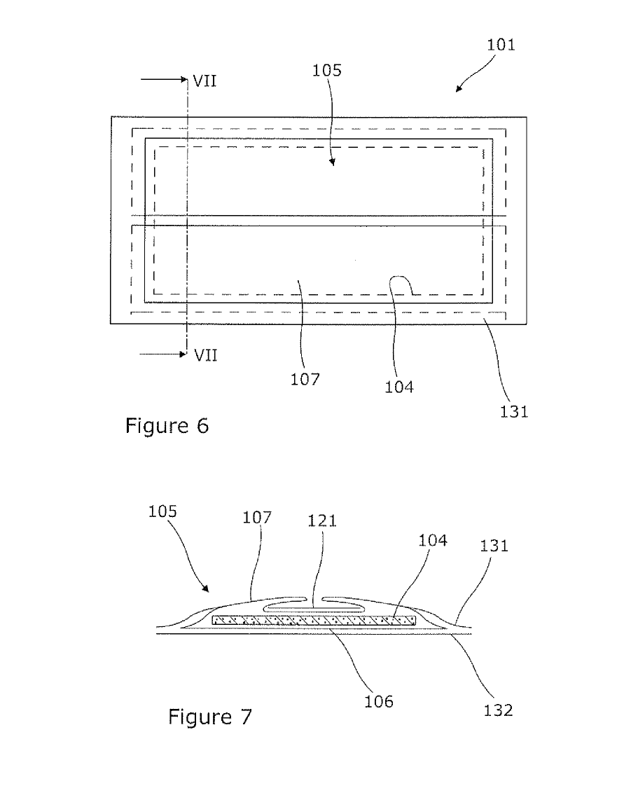

FIG. 6 is a plan view of an absorbent product of the invention in the form of a wound dressing; and

FIG. 7 is a cross-sectional view on the line VII-VII in FIG. 6.

Referring to the drawings, an absorbent product 1 is long and thin for placement, with others such products, on the floor 2 around the bottom of sides of an appliance such a freezer 3. Each product has an elongate matted strip 4 of absorbent material, including super absorber, within an enveloping container 5. This has a double skin bottom 6 and a single, folded top skin 7 and is heat sealed around its periphery 8 to contain the absorbent material. The top skin is of porous non-woven material, through which moisture dripping onto it can pass for absorption by the strip 4. The outer skin 61 of the bottom is of perforate plastics material film and the inner skin 62 is of non-woven material as the top is. Moisture on the floor can pass by capillary action into through the bottom. When the container has absorbed its fill of moisture and there is a tendency for the absorbent material to ooze out, the outer skin assists in resisting this oozing.

The non-woven sheet material of the top has an elongate, double Z fold 11 along its length. That is to say, outer webs 12 extend from the sealed periphery to the longitudinal middle of the top where they are folded back under themselves defining fold lines 14 where the outer webs abut. Immediately under the top webs, the sheet material extends hack from the fold lines 14, some 50% of the width of the outer webs, as intermediate webs 15 to inner fold lines 16. The sheet material extends inwards as inner webs 17, which adjoin beneath the outer fold lines. Thus it can be appreciated that outer, intermediate and inner webs for Z folds. A series of elongate pieces 18 of sheet material with self adhesive applied are applied across the fold lines 14, whereby the folds are held together. The pieces should be of material such as to not disintegrate merely from moisture yet able to resist only light tension in the top webs.

As the container absorbs moisture from condensation running down the freezer onto the floor, it expands from a flat form to a round shape. The super absorber causes tension in the skins of the container as it reaches its absorption limit. This tension tears the self-adhesive strips, which are liable to have been softened by moisture or pulls the strips from the outer skins. The result is that the folds unfold and the round shape slumps. In doing so the inner webs are exposed. An indicator 21 in the form off the words “FULL-CHANGE” printed in a bright colour repeatedly along the inner webs is provided to alert maintenance staff of the need to change filled absorbent products for fresh one.

Referring to FIGS. 6 & 7, another embodiment is shown in the form of a wound dressing 101, having its absorbent material 104 in a container 105 of non-woven material with a plain wound side 106 and a pleated/folded outer side 107, normally covering an indicator 121. The dressing has a peripheral web 131 of self adhesive film, covered by a release paper prior to use. In use, covering a wound, exudate from the latter is absorbed until the dressing has reached its capacity; whereupon the outer side expands and exposes the indicator, indicating that the dressing needs to be changed.

The invention is not intended to be restricted to the details of the above described embodiments. The double Z folds can be replaced by a single Z fold. The self-adhesive pieces can be replaced by adhesive or heat seal tacks of the webs. The Printed indicator can be replaced by a separate ribbon like piece exposed by the opening of the folds. The above two embodiments are rectangular. However, this can be different, for instance round of oblong in the case or a wound dressing. The absorbent material can include a biocidal agent, in particular in the case of a wound dressing to prevent the ingress of harmful substances which may cause infection in an open wound. The absorbent material and/or biocidal agent can be provided loose within the container or preferably can be provided as an integrated strip enclosed within the container.

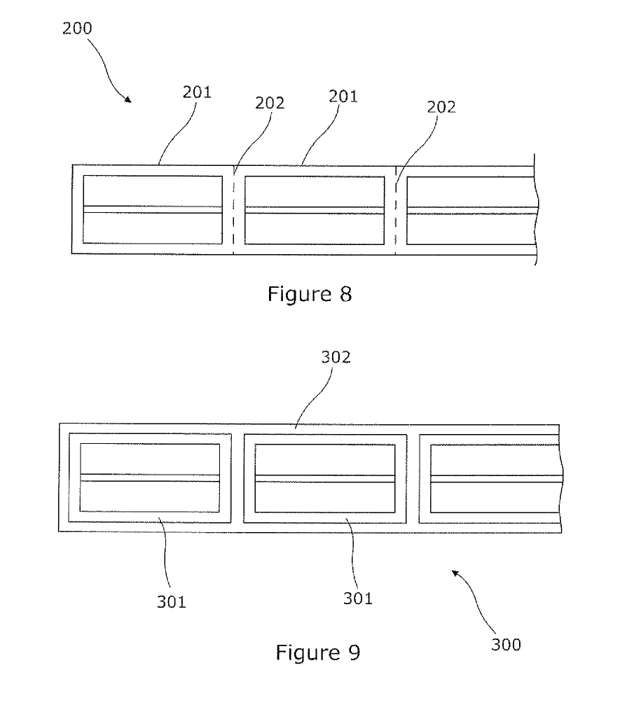

As shown in the embodiments of FIG. 9 and FIG. 10, a plurality of absorbent products may be aligned end to end to form an elongate string of absorbent products. A string of absorbent products may be stacked by folding adjacent products on top of each other or the string may be rolled up into a roll for storage.

Referring to FIG. 9, a plurality of absorbent products 201 are joined end to end to form a continuous elongate absorbent string 200. The non-woven sheet material is continuous between the ends of adjacent products 201, but is perforated along a line 202 for separation of individual products or a strip of a desired number of products from the ribbon.

Referring to FIG. 10, a plurality of absorbent products 301 are aligned on a strip of release paper 302 to form a string. The products 301 have an adhesive layer sprayed onto their bottom skin, which secures the products in place on the release paper until needed, whereby individual products may be peeled off the paper and installed on a surface for use.

1-23. (canceled)

24. An absorbent product comprising:
absorbent material and
a permeable container of sheet material containing the absorbent material and having:
a fold in the sheet material with an outer web of the material over-lying an inner web at the fold and
an indicator on the inner web
the arrangement being such that the outer web normally covers the indicator when the absorbent material has absorbed a determined amount of moisture, whereupon it swells, opening the fold and exposing the indicator.

25. An absorbent product according to claim 24, wherein the fold is a Z fold with an intermediate web also overlying the inner web, or wherein two Z folds are provided, one beside the other, providing an inner web with a greater extent, as two adjacent inner webs, than the extent of the individual outer webs.

26. An absorbent product according to claim 24, wherein the indicator is a printing on the inner web.

27. An absorbent product according to claim 25, wherein the indicator is a printing on the inner web and extends onto the intermediate web or webs.

28. An absorbent product according to claim 24, wherein the indicator is a separate element, such as a ribbon lying along the inner web.

29. An absorbent product according to claim 24, wherein the product further comprises:
i. frangible means holding the outer web over-lying the indicator on the inner web,
the frangible means normally holding the outer web in its position covering the indicator until expansion of the absorbent material on absorption of causes the frangible means to break and expose the indicator.

30. An absorbent product according to claim 29, wherein the frangible means is one or more adhesive elements applied to the outer web or webs to hold the fold or folds closed.

31. An absorbent product according to claim 29, wherein the frangible means is adhesive material on the inner web and/or indicator ribbon.

32. An absorbent product according to claim 29, wherein the frangible means is a series of tacks of arranged to hold the fold closed.

33. An absorbent product according to claim 24, wherein the permeable container is formed completely of non-woven fabric.

34. An absorbent product according to claim 24, wherein the permeable container is formed completely of perforate plastics material film.

35. An absorbent product according to claim 24, wherein the permeable container is formed of a combination of non-woven fabric and perforate plastics material film.

36. An absorbent product according to claim 24, wherein the permeable container comprises a top of non-woven fabric having the fold and a bottom is of perforate plastics material film.

37. An absorbent product according to claim 36, wherein the perforate film is provided as an outer layer to an inner layer of non-woven fabric.

38. An absorbent product according to claim 24, wherein the bottom has applied to it adhesive or non-slip material for retaining the absorbent product in a use position.

39. An absorbent product according to claim 24, wherein a self-adhesive web extends out from the edges of the container for its securement in a use position.

40. An absorbent product according to claim 24, wherein the product is adapted for absorbing exudate from a region covered by the container, such as a wound or a leak.

41. An absorbent product according to claim 24, wherein absorbent material includes or is completely super-absorber.

42. An absorbent product according to claim 41, wherein the super-absorber is provided loose.

43. An absorbent product according to claim 41, wherein the super-absorber is provided as an integrated strip of absorbent material enclosed within the container.

44. An absorbent product according to claim 24, wherein a biocidal agent is included with the absorbent material.
